(19) United States
(12) Patent Application Publication
(10) Pub. No.: US 2014/0194969 A1
(43) Pub. Date: Jul. 10, 2014

(54) HYDRAULICALLY ACTUATED HANDLE ASSEMBLY FOR MEDICAL DEVICE DEPLOYMENT SYSTEM

(71) Applicant: Josh Headley, Ellettsville, IN (US)

(72) Inventor: Josh Headley, Ellettsville, IN (US)

(21) Appl. No.: 13/737,292

(22) Filed: Jan. 9, 2013

(51) Int. Cl. A61F 2/966

(52) U.S. Cl. CPC A61F 2/966
USPC 623/1.12

(57) ABSTRACT

A medical device deployment system includes an inner shaft telescopically received within a retractable sheath and a medical device positioned over the inner shaft. A handle assembly houses a hollow washer and includes a hollow handle body having an open distal end receiving the inner shaft and the retractable sheath. An open proximal end of the retractable sheath is attached to the washer and the inner shaft is received through an opening of the washer. In a pre-deployment configuration, the washer has an initial position within the handle body, an expandable fluid chamber within the handle body has an initial volume, and the retractable sheath restricts radial expansion of the medical device. In a post-deployment configuration, the washer has a retracted position proximally spaced from the initial position, the fluid chamber has an expanded volume, and the retractable sheath is retracted and permits radial expansion of the medical device.

### TECHNICAL FIELD

The present disclosure relates generally to a handle assembly for a medical device deployment system, and more particularly to a handle assembly housing a hydraulically actuated hollow washer configured to retract a retractable sheath and deploy a medical device.

### BACKGROUND

Various medical devices, including stents, stent grafts, and venous filters, are deployed within the vasculature of a patient using deployment devices. Some of the medical devices are self-expanding, in a radial direction, and require restriction from radial expansion prior to deployment. According to some deployment systems, an outer sheath maintains a restricted position of the self-expanding medical device during advancement of the medical device to a deployment site. Once the medical device is positioned at or near the deployment site, the sheath is removed, or retracted, to permit radial expansion of the self-expanding medical device. The retraction of the sheath is typically facilitated through manipulation of a handle positioned at a proximal end of the deployment system.

Although a variety of different deployment system handles exist, ranging from relatively simple to relatively complex devices, a conventional pull-type handle includes a proximal handle portion and a distal handle portion. The proximal handle portion is configured to maintain a relatively stationary position of a pusher catheter, which supports the self-expanding medical device, while the distal handle portion is configured to retract a sheath positioned over the self-expanding medical device. Prior to deployment, the proximal and distal handle portions are positioned away from one another, resulting in a relatively long pre-deployment length of the deployment system. Deployment of the medical device is initiated by proximally retracting the distal handle portion, which is connected to the sheath, toward the proximal handle portion. In addition to having a relatively long pre-deployment length, the conventional pull-type handle requires non-intuitive manipulation of the handle portions, which may be difficult for less experienced clinicians. Further, the expanded pre-deployment configuration may be relatively unstable, particularly for the deployment of relatively large medical devices.

U.S. Pat. No. 6,113,608 to Monroe et al. discloses a stent delivery device having a hydraulically actuated retractable sheath. In particular, a pressurizing fluid is either supplied by an inflation lumen to a portion of a piston housing or is withdrawn from a portion of the piston housing to actuate a piston. As the piston moves, a retractable sheath, which is connected to the piston, moves as well and causes the sheath to retract. Although the handle of the Monroe et al. disclosure may be useful for some applications, it should be appreciated that there is a continuing need for efficient and effective handles for medical device deployment systems.

The present disclosure is directed toward one or more of the problems or issues set forth above.

### SUMMARY OF THE DISCLOSURE

In one aspect, a medical device deployment system includes an inner shaft having an elongate body, a proximal end, a distal end, and a medical device support region at the distal end of the elongate body. The inner shaft is telescopically received within a retractable sheath having an elongate tubular body defining a lumen extending from an open proximal end to an open distal end. A medical device is positioned over the inner shaft at the medical device support region. A handle assembly includes a hollow handle body defining a movement axis and having an open distal end receiving the inner shaft and the retractable sheath therethrough. A hollow washer is positioned within the hollow handle body and is movable along the movement axis. The open proximal end of the retractable sheath is attached to the hollow washer and the inner shaft is received through an opening defined by the hollow washer. The hollow handle body defines an expandable fluid chamber between the open distal end of hollow handle body and the hollow washer. A deployment port is positioned through the hollow handle body and is in fluid communication with the expandable fluid chamber. The medical device deployment system has a pre-deployment configuration in which the hollow washer has an initial position within the hollow handle body, the expandable fluid chamber has an initial volume, and the retractable sheath restricts radial expansion of the medical device. The medical device deployment system also has a post-deployment configuration in which the hollow washer has a retracted position proximally spaced from the initial position, the expandable fluid chamber has an expanded volume greater than the initial volume, and the retractable sheath is retracted such that the medical device is not restricted from radial expansion by the retractable sheath.

In another aspect, a method of deploying a medical device within a body lumen using the medical device deployment system described above is also provided. The method includes a step of advancing a distal portion of the medical device deployment system toward a deployment site within the body lumen with the medical device deployment system in the pre-deployment configuration, in which the hollow washer has an initial position within the hollow handle body, the expandable fluid chamber has an initial volume, and the retractable sheath restricts radial expansion of the medical device. The method also includes increasing the initial volume of the expandable fluid chamber toward an expanded volume by introducing fluid into the expandable fluid chamber through the deployment port. In response, the retractable sheath is proximally retracted by moving the hollow washer toward a retracted position proximally spaced from the initial position. The medical device is then radially expanded responsive to the proximal retraction of the retractable sheath.

### BRIEF DESCRIPTION OF THE DRAWINGS

FIG. 1 is a partially sectioned side diagrammatic view of a medical device deployment system, shown in a pre-deployment configuration, according to one embodiment of the present disclosure;

FIG. 2 is a partially sectioned side diagrammatic view of the medical device deployment system of FIG. 1, shown in a post-deployment configuration;

FIG. 3 is a side diagrammatic view of a vascular structure of a patient at one stage of a medical device deployment procedure using the medical device deployment system of the previous Figs.;

FIG. 4 is a side diagrammatic view of the vascular structure at another stage of the medical device deployment procedure; and

FIG. 5 is a side diagrammatic view of the vascular structure at another stage of the medical device deployment procedure.

### DETAILED DESCRIPTION

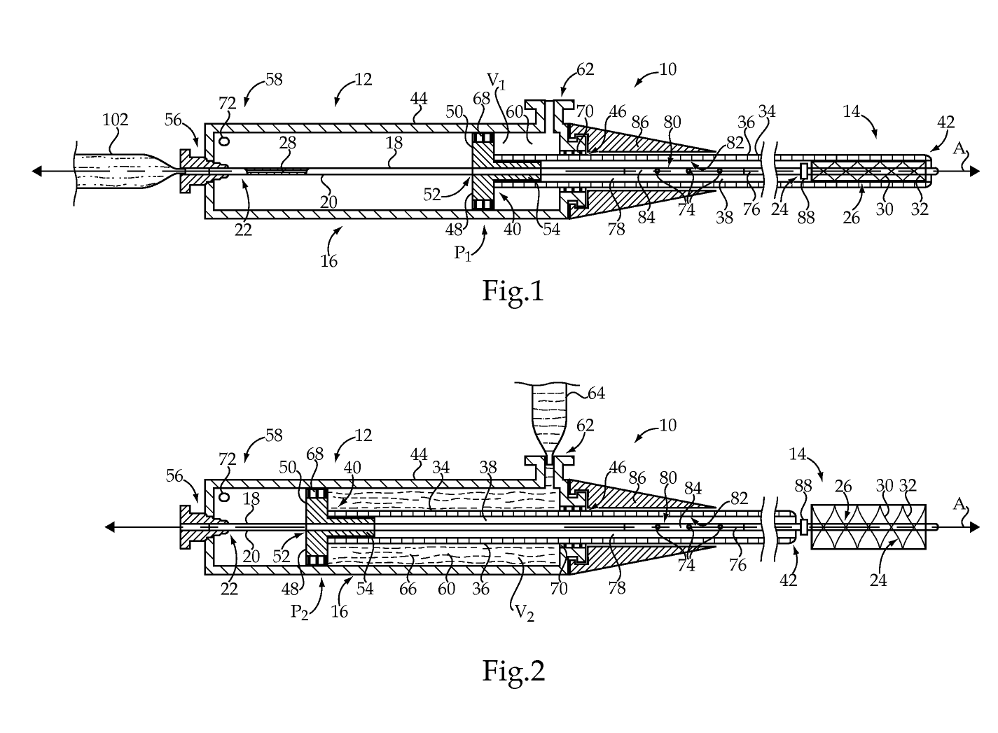

Referring to FIG. 1, there is shown a medical device deployment system 10 according to one embodiment of the present disclosure. The medical device deployment system 10 may include a number of components, which may be provided within a sterile, tear open package (not shown), as is known in the art. In performing a medical device deployment procedure on a patient, some or all of the components of the medical device deployment system 10 may be used, depending upon the specifics of the procedure to be performed. As should be appreciated, however, components of the medical device deployment system 10 might be separately packaged and/or the medical device deployment system 10 might also include components in addition to those shown, including components routinely used in percutaneous vascular procedures.

In general, the medical device deployment system 10 has a proximal end 12 and a distal end 14. As shown, a handle assembly 16, which may include relatively rigid components made from medical grade materials, is disposed at the proximal end 12. In the present disclosure, “proximal” will be used to refer to the end of a component or feature that is closest to a clinician, while “distal” is used to refer to a component or feature that is farthest away from the clinician. Such meanings are consistent with conventional use of the terms and, as such, should be understood by those skilled in the art.

According to the exemplary embodiment, the medical device deployment system 10 includes an inner shaft 18 having an elongate body 20, a proximal end 22, a distal end 24, and a medical device support region 26 at the distal end 24 of the elongate body 20. According to some embodiments, the inner shaft 18, which may include a hollow tubular body defining a lumen 28, may range in length from several inches to several feet long, and may have a catheter wall diameter that is orders of magnitude smaller than its length. The elongate body 20 may be made from any common medical tube material, such as, for example, a plastic, rubber, silicone, or Teflon material, and may exhibit both firmness and flexibility.

A medical device 30 may be positioned over the inner shaft 18 at the medical device support region 26. According to the exemplary embodiment, the medical device 30 may include a radially expanding stent 32 for providing tubular support within a blood vessel, canal, duct, or other bodily passageway. Radially expandable stents 32 are known and may be expanded using a balloon, or other known device, positioned at a distal portion of a delivery catheter. Alternatively, and according to the exemplary embodiment, the radially expanding stent 32 may be made from a resilient or shape memory material, such as, for example, nitinol, that is capable of self-expanding from a compressed state to an expanded state without the application of a radial force on the stent 32. Such a stent 32 may be referred to as a “self-expanding” stent 32. Although a self-expanding stent 32 will be discussed herein, those skilled in the art should appreciate that the medical device 30 may include alternative radially expandable prosthetic implants. For example, the medical device 30 may include a self-expanding, or otherwise expandable, stent graft or venous filter.

A retractable sheath 34 has an elongate tubular body 36 defining a lumen 38 extending from an open proximal end 40 to an open distal end 42. As shown, the inner shaft 18 is telescopically received within the retractable sheath 34. When the self-expanding stent 32, or other medical device, is loaded onto the inner shaft 18, the self-expanding stent 32 may be restricted from self-expansion using the elongate tubular sheath 34, which is slidably received over the elongate tubular body 20 of the inner shaft 18. According to this configuration, the retractable sheath 34 restricts radial expansion of the self-expanding stent 32 by contacting the stent 32 with an inner wall surface 82 defining the lumen 38 of the retractable sheath 34.

The handle assembly 16 includes a hollow handle body 44, which may be cylindrical in shape, defining a movement axis A. As shown, the inner shaft 18 and the retractable sheath 34 are received through an open distal end 46 of the hollow handle body 44. A hollow washer 48 is positioned within the hollow handle body 44 and is movable along the movement axis A. The hollow washer 48 may generally include a ring-shaped body 50 oriented perpendicularly relative to the movement axis A and defining an opening 52 substantially aligned with the movement axis A. The hollow washer 48 may also include an annular flange 54, or other similar structure, extending distally from the ring-shaped body 50. The open proximal end 40 of the retractable sheath 34 may be attached to the hollow washer 48 with the open proximal end 40 positioned about the annular flange 54. The open proximal end 40 may be frictionally engaged with, adhered, or otherwise attached to the hollow washer 48.

The inner shaft 18 may be slidably received through the opening 52 defined by the ring-shaped body 50 of the hollow washer 48. According to some embodiments, the proximal end 22 of the inner shaft 18 may be attached to a proximal port 56 positioned through a proximal end 58 of the hollow handle body 44. The proximal port 56 may provide access to the lumen 28 of the inner shaft 18 and may provide a structure for fixedly attaching the inner shaft 18 relative to the hollow handle body 44. It should be appreciated that connections or attachments between components of the medical device deployment system 10 may be made using any attachment means known to those skilled in the art.

The hollow handle body 44 also defines an expandable fluid chamber 60 between the open distal end 46 of the hollow handle body 44 and the hollow washer 48. A deployment port 62, in fluid communication with the expandable fluid chamber 60, may also be provided through the hollow handle body 44. A clinician may introduce a fluid, such as a liquid, into the expandable fluid chamber 60 through the deployment port 62 to increase or expand the expandable fluid chamber 60. In particular, the medical device deployment system 10 has a pre-deployment configuration, as shown in FIG. 1, in which the hollow washer 48 has an initial axial position P₁ within the hollow handle body 44, the expandable fluid chamber 60 has an initial volume V₁, and the retractable sheath 34 restricts radial expansion of the medical device 30. The medical device deployment system 10 also has a post-deployment configuration, as shown in FIG. 2, in which the hollow washer 48 has a retracted axial position P₂ proximally spaced from the initial axial position P₁, the expandable fluid chamber 60 has an expanded volume V₂ greater than the initial volume V₁, and the retractable sheath 34 is retracted such that the medical device 30 is not restricted from radial expansion by the retractable sheath 34. To be clear, the expandable fluid chamber 60 may be defined by a relatively rigid handle body 44 that utilizes movement of the hollow washer 48 to effect volumetric changes in the expandable fluid chamber 60. A preferable fluid for moving the hollow washer 48 may include a relatively incompressible fluid, such as a liquid.

Thus, as shown in FIG. 2, a clinician may fluidly attach a first syringe 64, such as via a luer fitting, at the deployment port 62 to introduce a fluid, such as a liquid 66, into the expandable fluid chamber 60. The liquid 66, which may include a readily available flushing media, such as saline, effectively deploys the medical device 30 by proximally moving the hollow washer 48 within the hollow handle body 44 and retracting the retractable sheath 34. To reduce leakage, a first sealing member 68 may be positioned about the hollow washer 48 and a second sealing member 70 may be positioned about the retractable sheath 34 at the open distal end 46 of the hollow handle body 44. The first and second sealing members 68 and 70 may be made from materials known to those skilled in the art and may be configured to substantially seal the expandable fluid chamber 60. A vent opening 72 may be provided through the hollow handle body 44 and may be sized to permit a desired amount of air to escape the hollow handle body 44. The vent opening 72 may be positioned proximally relative to the hollow washer 48 in both of the pre-deployment configuration (FIG. 1) and the post-deployment configuration (FIG. 2).

According to some embodiments, the inner shaft 18 may include a plurality of lateral openings 74 through walls 76 defining the elongate body 20 of the inner shaft 18. The plurality of lateral openings 74 may be positioned distally relative to the hollow washer 48 in both of the pre-deployment configuration of FIG. 1 and the post-deployment configuration of FIG. 2. These lateral openings 74 may facilitate simultaneous flushing of the lumen 28 of the inner shaft 18 and an intermediate region 78 defined by an exterior surface 80 of the inner shaft 18 and the inner surface 82 of the retractable sheath 34 by advancing fluid, such as a flushing media, through the proximal port 56, through the inner shaft 18, through the plurality of lateral openings 74, and through the intermediate region 78. According to some embodiments, a segment 84 of the elongate body 20 of the inner shaft 18 including the plurality of lateral openings 74 may include a metallic material, or other material having a higher durometer than the remaining portion of the inner shaft 18, for strengthening the inner shaft 18 at the openings 74. However, increasing a stiffness at the segment 84 is optional.

As shown, a distally tapered collar 86, or strain relief, may be supported from the open distal end 46 of the hollow handle body 44. The retractable sheath 34 may be telescopically received through the distally tapered collar 86. During use of the medical device deployment system 10, the distally tapered collar 86 may reduce excessive bending or kinking at the location where the inner shaft 18 and the retractable sheath 34 exit the open distal end 46 of the hollow handle body 44. Also, according to some embodiments, a pusher band 88 may be disposed on the exterior surface 80 of the inner shaft 18 proximal to the medical device 30 and may be configured to restrict proximal movement of the medical device 30 during relative movement of the inner shaft 18 and the retractable sheath 34. Additional features useful in medical device deployment systems may also be incorporated into the medical device deployment system 10.

### INDUSTRIAL APPLICABILITY

The present disclosure is generally applicable to medical device deployment systems. More specifically, the present disclosure is applicable to deployment systems for deploying self-expanding medical devices, such as stents, grafts, filters, and the like. Further, the present disclosure is applicable to handle assemblies for use with such medical device deployment devices.

Referring generally to FIGS. 1-5, an exemplary embodiment of a medical device deployment system 10 includes an inner shaft 18 telescopically received within a retractable sheath 34 and a medical device 30 positioned over the inner shaft 18. A handle assembly 16 houses a hollow washer 48 and includes a hollow handle body 44 having an open distal end 46 receiving the inner shaft 18 and the retractable sheath 34. An open proximal end 40 of the retractable sheath 34 is attached to the hollow washer 48 and the inner shaft 18 is received through an opening 52 of the hollow washer 48. Thus, the hollow washer 48 is configured to move the retractable sheath 34 while sliding along the inner shaft 18 within the hollow handle body 44. The hollow handle body 44 also defines an expandable fluid chamber 60 between the open distal end 46 of the hollow handle body 44 and the hollow washer 48. As will be described below, a clinician may introduce fluid, such as liquid 66, into the expandable fluid chamber 60 through a deployment port 62, which is in fluid communication with the expandable fluid chamber 60, to proximally move the hollow washer 48 and the retractable sheath 34.

Prior to use of the medical device deployment system 10, the clinician may simultaneously flush a lumen 28 of the inner shaft 18 and an intermediate region 78 defined by an exterior surface 80 of the inner shaft 18 and an inner surface 82 of the retractable sheath 34. In particular, a plurality of lateral openings 74 may be provided through walls 76 defining an elongate body 20 of the inner shaft 18 to facilitate the simultaneous flushing. For example, the clinician may advance a flushing media, such as saline, from a second syringe 102 (shown in FIG. 1) through a proximal port 56 of the handle assembly 16, through the inner shaft 18, through the plurality of lateral openings 74, and through the intermediate region 78. The flushing media may exit the medical device deployment system 10 through a distal end 14 of the system 10.

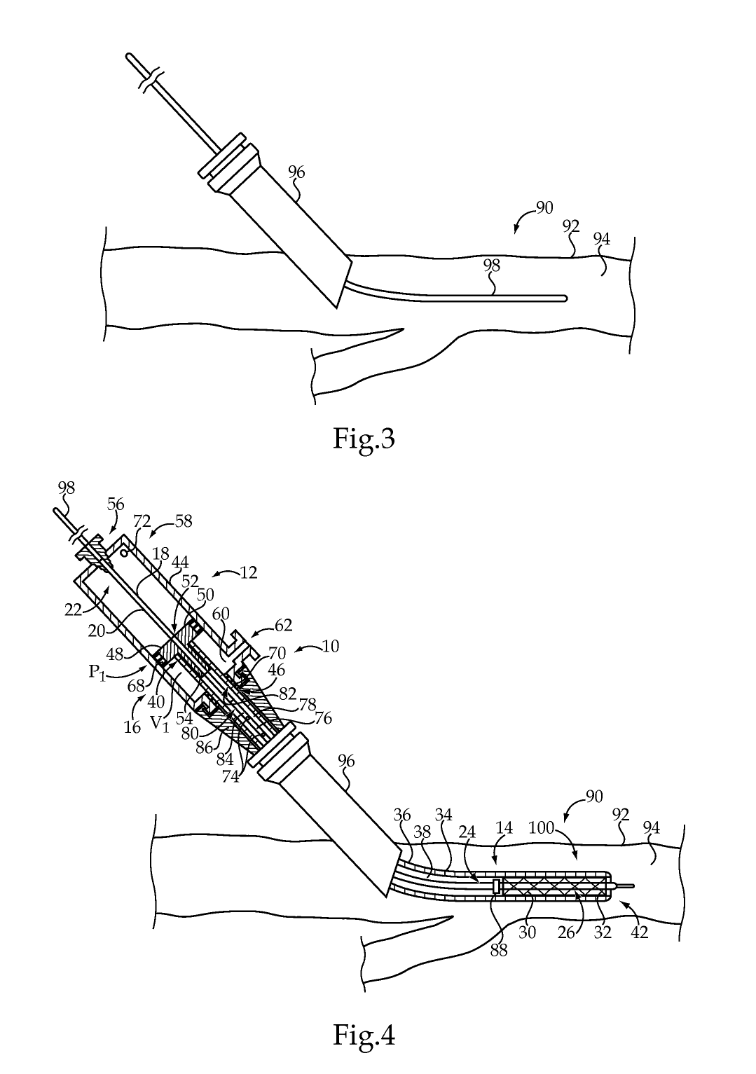

Referring specifically to FIGS. 3 through 5, a percutaneous vascular procedure using the medical device deployment system 10 of FIGS. 1 and 2 will be discussed with reference to a vascular structure 90 of a patient. The vascular structure 90, as should be appreciated, may include a vessel wall 92 defining a lumen 94. The clinician may first position a needle, or introducer 96, through the skin of a patient to gain access to the vascular structure 90. Thereafter, the clinician may insert a conventional wire guide 98 through a tube of the introducer 96 and into the vascular structure 90, as shown in FIG. 3.

Turning now to FIG. 4, the distal portion 14 of the medical device deployment system 10 may be inserted through the introducer 96 and over the wire guide 98. In particular, the hollow elongate body 20 of the inner shaft 18 may be advanced over the wire guide 98. The wire guide 98 may be received through the lumen 28 of the inner shaft 18 and may exit through the proximal port 56 through the hollow handle body 44. The distal portion 14 of the medical device deployment system 10 may be advanced toward a deployment site 100 within the body lumen 94 with the medical device deployment system 10 in a pre-deployment configuration, as described with respect to FIG. 1, in which the hollow washer 48 has an initial axial position P₁ within the hollow handle body 44, the expandable fluid chamber 60 has an initial volume V₁, and the retractable sheath 34 restricts radial expansion of the medical device 30.

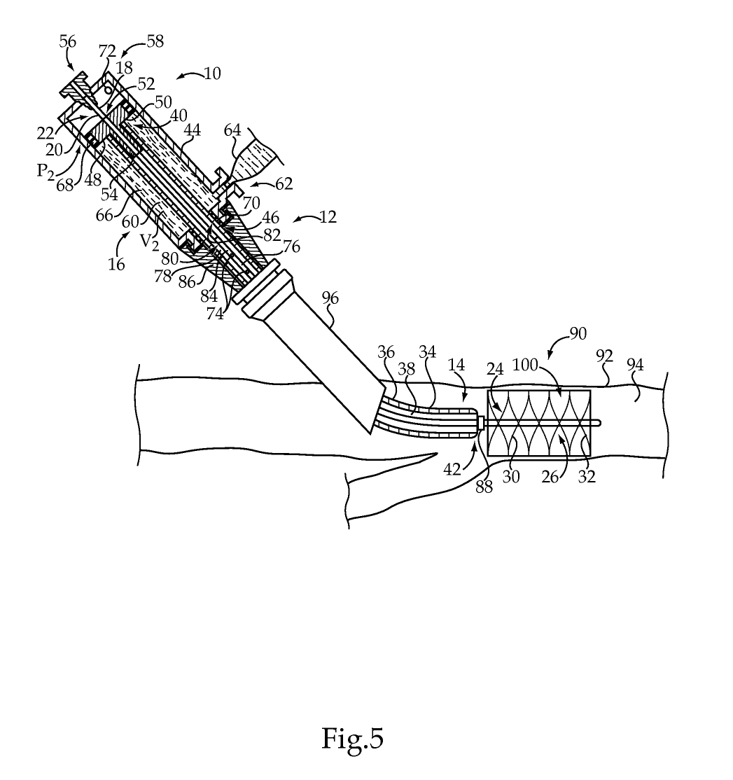

With the medical device deployment system 10 properly positioned relative to the deployment site 100, the retractable sheath 34 may be proximally retracted by proximally moving, or retracting, the hollow washer 48. In particular, the initial volume V₁ of the expandable fluid chamber 60 may be increased toward an expanded volume V₂ by introducing fluid into the expandable fluid chamber 60 through the deployment port 62, such as by using a syringe 64. As the fluid, which may be a liquid 66, enters the expandable fluid chamber 60, the fluid acts on the hollow washer 48 and expands the expandable fluid chamber 60 by moving the hollow washer 48 toward a retracted position P₂ proximally spaced from the initial position P₁. The retractable sheath 34, which is attached to the hollow washer 48, retracts with the hollow washer 48. As described above, a pusher band 88 may be disposed on the exterior surface 80 of the inner shaft 18 proximal to the medical device 30 for restricting proximal movement of the medical device 30 while the retractable sheath 34 is being retracted. The medical device 30, which may include a self-expanding stent 32, may be radially expanded responsive to the retraction of the retractable sheath 34, as shown in FIG. 5.

After deployment of the medical device 30, the medical device deployment system 10 may be withdrawn from the body lumen 94 with the medical device deployment system 10 in a post-deployment configuration, as described with reference to FIG. 2. According to the post-deployment configuration, the hollow washer 48 has a retracted axial position P₂ proximally spaced from the initial axial position P₁, the expandable fluid chamber 60 has an expanded volume V₂ greater than the initial volume V₁, and the retractable sheath 34 is retracted such that the medical device 30 is not restricted from radial expansion by the retractable sheath 34.

The medical device deployment system 10 described herein provides an effective means for deploying medical devices, such as medical device 30, of various sizes and configurations. Further, the handle assembly 16 of the medical device deployment system 10 requires fewer components than some conventional deployment devices, thus reducing cost and complexity. Yet further, the handle assembly 16 disclosed herein does not require an inflation lumen, which might occupy much needed space, for increasing a volume of the expandable fluid chamber 60.

It should be understood that the above description is intended for illustrative purposes only, and is not intended to limit the scope of the present disclosure in any way. Thus, those skilled in the art will appreciate that other aspects of the disclosure can be obtained from a study of the drawings, the disclosure and the appended claims.

What is claimed is:

1. A medical device deployment system, comprising:
an inner shaft having an elongate body, a proximal end, a distal end, and a medical device support region at the distal end of the elongate body;
a retractable sheath having an elongate tubular body defining a lumen extending from an open proximal end to an open distal end, wherein the inner shaft is telescopically received within the retractable sheath;
a medical device positioned over the inner shaft at the medical device support region; and
a handle assembly including:
a hollow handle body defining a movement axis and having an open distal end receiving the inner shaft and the retractable sheath therethrough;
a hollow washer positioned within the hollow handle body and movable along the movement axis, wherein the open proximal end of the retractable sheath is attached to the hollow washer and the inner shaft is received through an opening defined by the hollow washer;
an expandable fluid chamber within the hollow handle body between the open distal end of the hollow handle body and the hollow washer; and
a deployment port through the hollow handle body in fluid communication with the expandable fluid chamber;
wherein the medical device deployment system has a pre-deployment configuration in which the hollow washer has an initial position within the hollow handle body, the expandable fluid chamber has an initial volume, and the retractable sheath restricts radial expansion of the medical device, and a post-deployment configuration in which the hollow washer has a retracted position proximally spaced from the initial position, the expandable fluid chamber has an expanded volume greater than the initial volume, and the retractable sheath is retracted such that the medical device is not restricted from radial expansion by the retractable sheath.

2. The medical device deployment system of claim 1, further including a first sealing member positioned about the hollow washer and a second sealing member positioned about the retractable sheath at the open distal end of the hollow handle body, wherein the first and second sealing members are configured to seal the expandable fluid chamber.

3. The medical device deployment system of claim 2, wherein the elongate body of the inner shaft is hollow.

4. The medical device deployment system of claim 3, further including a proximal port through a proximal end of the hollow handle body, wherein the proximal end of the inner shaft is attached to the proximal port.

5. The medical device deployment system of claim 4, further including a plurality of lateral openings through walls defining the elongate body of the inner shaft, wherein the plurality of lateral openings are positioned distally relative to the hollow washer in both of the pre-deployment configuration and the post-deployment configuration.

6. The medical device deployment system of claim 5, wherein a segment of the elongate body of the inner shaft including the plurality of lateral openings includes a metallic material.

7. The medical device deployment system of claim 1, further including a distally tapered collar supported from the open distal end of the hollow handle body, wherein the retractable sheath is telescopically received through the distally tapered collar.

8. The medical device deployment system of claim 1, further including a vent opening through the hollow handle body, wherein the vent opening is positioned proximally relative to the hollow washer in both of the pre-deployment configuration and the post-deployment configuration.

9. The medical device deployment system of claim 1, wherein the medical device is a stent.

10. The medical device deployment system of claim 1, wherein the medical device is a self-expanding stent.

11. The medical device deployment system of claim 1, further including a pusher band disposed on an exterior surface of the inner shaft proximal to the medical device and configured to restrict proximal movement of the medical device during relative movement of the inner shaft and the retractable sheath.

12. A method of deploying a medical device within a body lumen using a medical device deployment system, wherein the medical device deployment system includes: an inner shaft having an elongate body, a proximal end, a distal end, and a medical device support region at the distal end of the elongate body; a retractable sheath having an elongate tubular body defining a lumen extending from an open proximal end to an open distal end, wherein the inner shaft is telescopically received within the retractable sheath; the medical device being positioned over the inner shaft at the medical device support region; and a handle assembly including a hollow handle body defining a movement axis and having an open distal end receiving the inner shaft and the retractable sheath therethrough, a hollow washer positioned within the hollow handle body and movable along the movement axis, wherein the open proximal end of the retractable sheath is attached to the hollow washer and the inner shaft is received through an opening defined by the hollow washer, an expandable fluid chamber within the hollow handle body between the open distal end of the hollow handle body and the hollow washer, and a deployment port through the hollow handle body in fluid communication with the expandable fluid chamber, the method comprising steps of:
advancing a distal portion of the medical device deployment system toward a deployment site within the body lumen with the medical device deployment system in a pre-deployment configuration in which the hollow washer has an initial position within the hollow handle body, the expandable fluid chamber has an initial volume, and the retractable sheath restricts radial expansion of the medical device;
increasing the initial volume of the expandable fluid chamber toward an expanded volume by introducing fluid into the expandable fluid chamber through the deployment port;
proximally retracting the retractable sheath by moving the hollow washer toward a retracted position proximally spaced from the initial position responsive to the increasing step; and
radially expanding the medical device responsive to the proximally retracting step.

13. The method of claim 12, further including sealing the expandable fluid chamber using a first sealing member positioned about the hollow washer and a second sealing member positioned about the retractable sheath at the open distal end of the hollow handle body.

14. The method of claim 13, wherein the advancing step includes advancing a hollow elongate body of the inner shaft over a wire guide, wherein the wire guide is received through a lumen of the inner shaft and exits through a proximal port through a proximal end of the hollow handle body, wherein the proximal end of the inner shaft is attached to the proximal port.

15. The method of claim 14, further including simultaneously flushing the inner shaft and an intermediate region defined by an exterior surface of the inner shaft and an inner surface of the retractable sheath by advancing fluid through the proximal port, through the inner shaft, through a plurality of lateral openings through walls defining the elongate body of the inner shaft, and through the intermediate region.

16. The method of claim 12, wherein the radially expanding step includes radially expanding a stent.

17. The method of claim 12, wherein the radially expanding step includes radially expanding a self-expanding stent.

18. The method of claim 12, further including restricting proximal movement of the medical device during the proximally retracting step using a pusher band disposed on an exterior surface of the inner shaft proximal to the medical device.

19. The method of claim 12, further including inserting the distal portion of the medical device deployment system through an introducer prior to the advancing step.
